(19) United States
(12) Patent Application Publication
(10) Pub. No.: US 2010/0197974 A1
(43) Pub. Date: Aug. 5, 2010

(54) PROCESSES FOR MAKING DIALKYL ETHERS FROM ALCOHOLS

(75) Inventors: Mark Andrew Harmer, Landenberg, PA (US); Michael B. D'Amore, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DU PONT DE NEMOURS AND COMPANY, Wilmington, DE (US)

(21) Appl. No.: 12/676,160

(22) PCT Filed: Sep. 5, 2008
(86) PCT No.: PCT/US08/75302

§ 371 Date: Mar. 3, 2010

Related U.S. Application Data
(60) Provisional application No. 60/970,081, filed on Sep. 5, 2007.

(51) Int. Cl. C07C 41/01

(52) U.S. Cl. 568/671

(57) ABSTRACT

Processes for the preparation of dialkyl ethers from C₄ to C₈ straight-chain alcohols using an ionic liquid.

This application claims priority from, and the benefit of, U.S. Provisional Application No. 60/970,081, filed Sep. 5, 2007, which is by this reference incorporated in its entirety as a part hereof for all purposes.

### TECHNICAL FIELD

This invention is concerned with processes for the preparation of dialkyl ethers from straight-chain alcohols.

### BACKGROUND

Ethers such as dibutyl ether are useful as solvents and as diesel fuel cetane enhancers. See, for example, Kotrba, “Ahead of the Curve”, Ethanol Producer Magazine, November 2005; and WO 01/18154, wherein an example of a diesel fuel formulation comprising dibutyl ether is disclosed.

The production of ethers from alcohol, such as the production of dibutyl ether from butanol, is known and is generally described in Kara et al, Kirk-Othmer Encyclopedia of Chemical Technology, Fifth Ed., Vol. 10, Section 5.3, pp. 567˜583. The reaction is generally carried out via the dehydration of an alcohol by sulfuric acid, or by catalytic dehydration over ferric chloride, copper sulfate, silica, or silica-alumina at high temperatures. Bringue et al [J. Catalysis (2006) 244:33-42] disclose thermally stable ion-exchange resins for use as catalysts for the dehydration of 1-pentanol to di-n-pentyl ether. WO 07/38360 discloses a method for making polytrimethylene ether glycols in the presence of an ionic liquid.

A need nevertheless remains for commercially-advantageous processes to prepare ethers from alcohols.

### SUMMARY

The inventions disclosed herein include processes for the preparation of dialkyl ethers from alcohols, the use of such processes, and the products obtained and obtainable by such processes.

Features of certain of the processes of this invention are described herein in the context of one or more specific embodiments that combine various such features together. The scope of the invention is not, however, limited by the description of only certain features within any specific embodiment, and the invention also includes (1) a subcombination of fewer than all of the features of any described embodiment, which subcombination may be characterized by the absence of the features omitted to form the subcombination; (2) each of the features, individually, included within the combination of any described embodiment; and (3) other combinations of features formed by grouping only selected features of two or more described embodiments, optionally together with other features as disclosed elsewhere herein. Some of the specific embodiments of the processes hereof are as follows:

In the processes disclosed herein, a dialkyl ether is prepared in a reaction mixture by (a) contacting at least one C₄ to C₈ straight-chain alcohol with at least one homogeneous acid catalyst in the presence of at least one ionic liquid to form (i) a dialkyl ether phase of the reaction mixture that comprises a dialkyl ether, and (ii) an ionic liquid phase of the reaction mixture; and (b) separating the dialkyl ether phase of the reaction mixture from the ionic liquid phase of the reaction mixture to recover a dialkyl ether product; wherein an ionic liquid is represented by the structure of the Formula Z⁺A⁻ as set forth below.

Ethers, such as the dialkyl ethers produced by the processes hereof, are useful as solvents, plasticizers and as additives in transportation fuels such as gasoline, diesel fuel and jet fuel.

### BRIEF DESCRIPTION OF THE DRAWING

FIG. 1 shows vials in which dibutyl ether is formed from 1-butanol. The vial on the left (labeled “Reactants) contains 1-butanol, a homogeneous acid catalyst and an ionic liquid. The vial on the right (labeled “Products”) contains, above the line indicated by the arrow, a phase (labeled “Dibutyl ether”) in which the dibutyl ether product resides, and contains below the line a phase (labeled “IL/acid”) in which an ionic liquid and an acid catalyst or catalyst residue reside.

### DETAILED DESCRIPTION

There are herein disclosed processes for preparing dialkyl ethers in the presence of at least one ionic liquid and at least one acid catalyst. Where a homogeneous acid catalyst is used, these processes provide an advantage in that the product dialkyl ether can be recovered in a product phase that is separate from an ionic liquid phase that contains an ionic liquid and an acid catalyst.

In the description of the processes hereof, the following definitional structure is provided for certain terminology as employed in various locations in the specification:

An “alkane” or “alkane compound” is a saturated hydrocarbon having the general formula CₙH₂ₙ₊₂, and may be a straight-chain, branched or cyclic compound.

An “alkene” or “alkene compound” is an unsaturated hydrocarbon that contains one or more carbon-carbon double bonds, and may be a straight-chain, branched or cyclic compound.

An “alkoxy” radical is a straight-chain or branched alkyl group bound via an oxygen atom.

An “alkyl” radical is a univalent group derived from an alkane by removing a hydrogen atom from any carbon atom:

—CₙH₂ₙ₊₁ where n=1. The alkyl radical may be a C₁˜C₂₀ straight-chain, branched or cycloalkyl radical. Examples of suitable alkyl radicals include methyl, ethyl, n-propyl,
i-propyl, n-butyl, t-butyl, n-pentyl, n-hexyl, cyclohexyl,
n-octyl, trimethylpentyl, and cyclooctyl radicals.

An “aromatic” or “aromatic compound” includes benzene and compounds that resemble benzene in chemical behavior.

An “aryl” radical is a univalent group whose free valence is to a carbon atom of an aromatic ring. The aryl moiety may contain one or more aromatic rings and may be substituted by inert groups, i.e. groups whose presence does not interfere with the reaction.

Examples of suitable aryl groups include phenyl, methylphenyl, ethylphenyl, n-propylphenyl, n-butylphenyl, t-butylphenyl, biphenyl, naphthyl and ethylnaphthyl radicals.

A “fluoroalkoxy” radical is an alkoxy radical in which at least one hydrogen atom is replaced by a fluorine atom.

A “fluoroalkyl” radical is an alkyl radical in which at least one hydrogen atom is replaced by a fluorine atom.

A “halogen” is a bromine, iodine, chlorine or fluorine atom.

A “heteroalkyl” radical is an alkyl group having one or more heteroatoms.

A “heteroaryl” radical is an aryl group having one or more heteroatoms.

A “heteroatom” is an atom other than carbon in the structure of a radical.

“Optionally substituted with at least one member selected from the group consisting of”, when referring to an alkane, alkene, alkoxy, alkyl, aryl, fluoroalkoxy, fluoroalkyl, heteroalkyl, heteroaryl, perfluoroalkoxy, or perfluoroalkyl radical or moiety, means that one or more hydrogens on a carbon chain of the radical or moiety may be independently substituted with one or more of the members of a recited group of substituents. For example, an optionally substituted —C₂H₅ radical or moiety may, without limitation, be —CF₂CF₃, —CH₂CH₂OH or —CF₂CF₂I where the group of substituents consist of F, I and OH.

A “perfluoroalkoxy” radical is an alkoxy radical in which all hydrogen atoms are replaced by fluorine atoms.

A “perfluoroalkyl” radical is an alkyl radical in which all hydrogen atoms are replaced by fluorine atoms.

In the processes disclosed herein, a dialkyl ether is prepared in a reaction mixture by (a) contacting at least one C₄ to C₈ straight-chain alcohol with at least one homogeneous acid catalyst in the presence of at least one ionic liquid to form (i) a dialkyl ether phase of the reaction mixture that comprises a dialkyl ether, and (ii) an ionic liquid phase of the reaction mixture; and (b) separating the dialkyl ether phase of the reaction mixture from the ionic liquid phase of the reaction mixture to recover a dialkyl ether product; wherein an ionic liquid is represented by the structure of the Formula Z⁺A⁻ as set forth below.

Suitable alcohols for use herein to prepare a dialkyl ether include straight-chain alcohols such as n-butanol, n-pentanol, n-hexanol, n-heptanol and n-octanol. A dialkyl ether as prepared by a process hereof may thus be a di-n-alkyl ether, but it may also be an ether in which one or both of the carbon chains thereon are derived from the same or different isomers of a C₄ to C₈ straight-chain alcohol. For example, where n-butanol is used as the alcohol reactant, one or both butyl moieties of the dialkyl ether product can independently be 1-butyl, 2-butyl, t-butyl or isobutyl.

In the ionic liquid of Formula Z⁺A⁻, Z⁺ is a cation selected from the group consisting of:

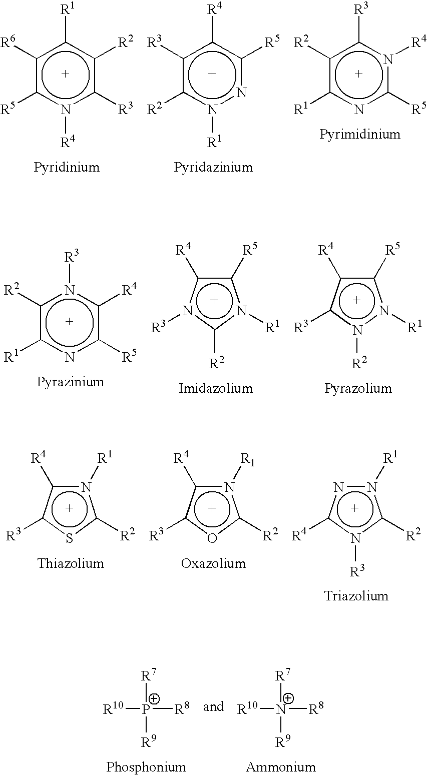

wherein R¹, R², R³, R⁴, R⁵ and R⁶ are independently selected from the group consisting of:
(i) H
(ii) halogen
(iii) —CH₃, —C₂H₅, or C₃ to C₂₅, preferably C₃ to C₂₀, straight-chain, branched or cyclic alkane or alkene, optionally substituted with at least one member selected from the group consisting of Cl, Br, F, I, OH, NH₂ and SH;
(iv) —CH₃, —C₂H₅, or C₃ to C₂₅, preferably C₃ to C₂₀, straight-chain, branched or cyclic alkane or alkene comprising one to three heteroatoms selected from the group consisting of O, N, Si and S, and optionally substituted with at least one member selected from the group consisting of Cl, Br, F, I, OH, NH₂ and SH;
(v) C₆ to C₂₅ unsubstituted aryl or unsubstituted heteroaryl having one to three heteroatoms independently selected from the group consisting of O, N, Si and S; and
(vi) C₆ to C₂₅ substituted aryl or substituted heteroaryl having one to three heteroatoms independently selected from the group consisting of O, N, Si and S; and wherein said substituted aryl or substituted heteroaryl has one to three substituents independently selected from the group consisting of
   (1) —CH₃, —C₂H₅, or C₃ to C₂₅, preferably C₃ to C₂₀, straight-chain, branched or cyclic alkane or alkene, optionally substituted with at least one member selected from the group consisting of Cl, Br, F, I, OH, NH₂ and SH,
   (2) OH,
   (3) NH₂, and
   (4) SH;
      R⁷, R⁸, R⁹, and R¹⁰ are independently selected from the group consisting of:
(vii) —CH₂, —C₂H₅, or C₃ to C₂₅, preferably C₃ to C₂₀, straight-chain, branched or cyclic alkane or alkene, optionally substituted with at least one member selected from the group consisting of Cl, Br, F, I, OH, NH₂ and SH;
(viii) —CH₃, —C₂H₅, or C₃ to C₂₅, preferably C₃ to C₂₀, straight-chain, branched or cyclic alkane or alkene comprising one to three heteroatoms selected from the group consisting of O, N, Si and S, and optionally substituted with at least one member selected from the group consisting of Cl, Br, F, I, OH, NH₂ and SH;
(ix) C₆ to C₂₅ unsubstituted aryl, or C₃ to C₂₅ unsubstituted heteroaryl having one to three heteroatoms independently selected from the group consisting of O, N, Si and S; and
(x) C₆ to C₂₅ substituted aryl, or C₃ to C₂₅ substituted heteroaryl having one to three heteroatoms independently selected from the group consisting of O, N, Si and S; and wherein said substituted aryl or substituted heteroaryl has one to three substituents independently selected from the group consisting of
   (1) —CH₃, —C₂H₅, or C₃ to C₂₅, preferably C₃ to C₂₀, straight-chain, branched or cyclic alkane or alkene, optionally substituted with at least one member selected from the group consisting of Cl, Br, F, I, OH, NH₂ and SH,
   (2) OH,
   (3) NH₂, and
   (4) SH;
      wherein optionally at least two of R¹, R², R³, R⁴, R⁵, R⁶, R⁷, R⁸, R⁹, and R¹⁰ can together form a cyclic or bicyclic alkanyl or alkenyl group; and

A⁻ is an anion selected from the group consisting of R¹¹—SO₂⁻ and (R¹²—SO₂)₂N⁻; wherein R¹¹ and R¹² are independently selected from the group consisting of:
(a) —CH₃, —C₂H₅, or C₃ to C₂₅, preferably C₃ to C₂₀, straight-chain, branched or cyclic alkane or alkene, optionally substituted with at least one member selected from the group consisting of Cl, Br, F, I, OH, NH₂ and SH;
(b) —CH₃, —C₂H₅, or C₃ to C₂₅, preferably C₃ to C₂₀, straight-chain, branched or cyclic alkane or alkene comprising one to three heteroatoms selected from the group consisting of O, N, Si and S, and optionally substituted with at least one member selected from the group consisting of Cl, Br, F, I, OH, NH₂ and SH;
(c) C₆ to C₂₅ unsubstituted aryl or unsubstituted heteroaryl having one to three heteroatoms independently selected from the group consisting of O, N, Si and S; and
(d) C₆ to C₂₅ substituted aryl or substituted heteroaryl having one to three heteroatoms independently selected from the group consisting of O, N, Si and S; and wherein said substituted aryl or substituted heteroaryl has one to three substituents independently selected from the group consisting of:
   (1) —CH₃, —C₂H₅, or C₃ to C₂₅, preferably C₃ to C₂₀, straight-chain, branched or cyclic alkane or alkene, optionally substituted with at least one member selected from the group consisting of Cl, Br, F, I, OH, NH₂ and SH,
   (2) OH,
   (3) NH₂, and
   (4) SH.

In one embodiment, the anion A⁻ is selected from the group consisting of: [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, and [(CF₂HCF₂SO₂)₂N]⁻, and [(CF₃CFHCF₂SO₂)₂N]⁻.

In another embodiment, an ionic liquid is selected from the group consisting of 1-butyl-2,3-dimethylimidazolium 1,1,2,2-tetrafluoroethanesulfonate, 1-butyl-methylimidazolium 1,1,2,2-tetrafluoroethanesulfonate, 1-ethyl-3-methylimidazolium 1,1,2,2-tetrafluoroethanesulfonate, 1-ethyl-3-methylimidazolium 1,1,2,3,3,3-hexafluoropropanesulfonate, 1-hexyl-3-methylimidazolium 1,1,2,2-tetrafluoroethanesulfonate, 1-dodecyl-3-methylimidazolium 1,1,2,2-tetrafluoroethanesulfonate, 1-hexadecyl-3-methylimidazolium 1,1,2,2-tetrafluoroethanesulfonate, 1-octadecyl-3-methylimidazolium 1,1,2,2-tetrafluoroethanesulfonate, N-(1,1,2,2-tetrafluoroethyl)propylimidazole 1,1,2,2-tetrafluoroethanesulfonate, N-(1,1,2,2-tetrafluoroethyl)ethylperfluorohexylimidazole 1,1,2,2-tetrafluoroethanesulfonate, 1-butyl-3-methylimidazolium 1,1,2,3,3,3-hexafluoropropanesulfonate, 1-butyl-3-methylimidazolium 1,1,2-trifluoro-2-(trifluoromethoxy)ethanesulfonate, 1-butyl-3-methylimidazolium 1,1,2-trifluoro-2-(perfluoroethoxy)ethanesulfonate, tetradecyl(tri-n-hexyl)phosphonium 1,1,2-trifluoro-2-(perfluoroethoxy)ethanesulfonate, tetradecyl(tri-n-butyl)phosphonium 1,1,2,3,3,3-hexafluoropropanesulfonate, tetradecyl(tri-n-hexyl)phosphonium 1,1,2-trifluoro-2-(trifluoromethoxy)ethanesulfonate, 1-ethyl-3-methylimidazolium 1,1,2,2-tetrafluoro-2-(pentafluoroethoxy)sulfonate, (3,3,4,4,5,5,6,6,7,7,8,8,8-tridecafluorooctyl)-trioctylphosphonium 1,1,2,2-tetrafluoroethanesulfonate, 1-methyl-3-(3,3,4,4,5,5,6,6,7,7,8,8,8-tridecafluorooctyl)imidazolium 1,1,2,2-tetrafluoroethanesulfonate, and tetra-n-butylphosphonium 1,1,2-trifluoro-2-(perfluoroethoxy)ethanesulfonate.

Ionic liquids are organic compounds that are liquid at room temperature (approximately 25° C.). They differ from most salts in that they have very low melting points, they tend to be liquid over a wide temperature range, and have been shown to have high heat capacities. Ionic liquids have essentially no vapor pressure, and they can either be neutral, acidic or basic. The properties of an ionic liquid will show some variation according to the identity of the cation and anion. However, a cation or anion of an ionic liquid useful for this invention can in principle be any cation or anion such that the cation and anion together form an organic salt that is fluid at or below about 100° C.

Many ionic liquids are formed by reacting a nitrogen-containing heterocyclic ring, preferably a heteroaromatic ring, with an alkylating agent (for example, an alkyl halide) to form a quaternary ammonium salt, and performing ion exchange or other suitable reactions with various Lewis acids or their conjugate bases to form the ionic liquid. Examples of suitable heteroaromatic rings include substituted pyridines, imidazole, substituted imidazole, pyrrole and substituted pyrroles. These rings can be alkylated with virtually any straight, branched or cyclic C₁₋₂₀ alkyl group, but preferably, the alkyl groups are C₁₋₁₆ groups, since groups larger than this may produce low melting solids rather than ionic liquids. Various triarylphosphines, thioethers and cyclic and non-cyclic quaternary ammonium salts may also been used for this purpose. Counterions that may be used include chloroaluminate, bromoaluminate, gallium chloride, tetrafluoroborate, tetrachloroborate, hexafluorophosphate, nitrate, trifluoromethane sulfonate, methylsulfonate, p-toluenesulfonate, hexafluoroantimonate, hexafluoroarsenate, tetrachloroaluminate, tetrabromoaluminate, perchlorate, hydroxide anion, copper dichloride anion, iron trichloride anion, zinc trichloride anion, as well as various lanthanum, potassium, lithium, nickel, cobalt, manganese, and other metal-containing anions.

Ionic liquids may also be synthesized by salt metathesis, by an acid-base neutralization reaction or by quaternizing a selected nitrogen-containing compound; or they may be obtained commercially from several companies such as Merck (Darmstadt, Germany) or BASF (Mount Olive, N.J.).

Representative examples of ionic liquids useful herein included among those that are described in sources such as J. Chem. Tech. Biotechnol., 68:351-356 (1997); Chem. Ind., 68:249-263 (1996); J. Phys. Condensed Matter, 5: (supp 34B):B99-B106 (1993); Chemical and Engineering News, Mar. 30, 1998, 32-37; J. Mater. Chem., 8:2627-2636 (1998); Chem. Rev., 99:2071-2084 (1999); and WO 05/113,702 (and references therein cited). In one embodiment, a library, i.e. a combinatorial library, of ionic liquids may be prepared, for example, by preparing various alkyl derivatives of a quaternary ammonium cation, and varying the associated anions. The acidity of the ionic liquids can be adjusted by varying the molar equivalents and type and combinations of Lewis acids.

Cations of ionic liquids useful herein are available commercially or may be synthesized by known methods. The fluoroalkyl sulfonate anions may be synthesized from perfluorinated terminal olefins or perfluorinated vinyl ethers generally according to the method of Koshar et al [J. Am. Chem. Soc. (1953) 75:4595-4596]; in one embodiment, sulfite and bisulfite are used as the buffer in place of bisulfite and borax, and in another embodiment, the reaction is carried out in the absence of a radical initiator. 1,1,2,2-Tetrafluoroethanesulfonate, 1,1,2,3,3,3-hexafluoropropanesulfonate, 1,1,2-trifluoro-2-(trifluoromethoxy)ethanesulfonate, and 1,1,2-trifluoro-2-(pentafluoroethoxy)ethanesulfonate may be synthesized according to a modified version of Koshar (supra). Preferred modifications include using a mixture of sulfite and bisulfite as the buffer, freeze drying or spray drying to isolate the crude 1,1,2,2-tetrafluoroethanesulfonate and 1,1,2,3,3,3-hexafluoropropanesulfonate products from the aqueous reaction mixture, using acetone to extract the crude 1,1,2,2-tetrafluoroethanesulfonate and 1,1,2,3,3,3-hexafluoropropanesulfonate salts, and crystallizing 1,1,2-trifluoro-2-(trifluoromethoxy)ethanesulfonate and 1,1,2-trifluoro-2-(pentafluoroethoxy)ethanesulfonate from the reaction mixture by cooling.

Other ionic liquids suitable for use herein may be made as follows: A first solution is made by dissolving a known amount of the halide salt of the cation in deionized water. This may involve heating to ensure total dissolution. A second solution is made by dissolving an approximately equimolar amount (relative to the cation) of the potassium or sodium salt of the anion in deionized water. This may also involve heating to ensure total dissolution. Although it is not necessary to use equimolar quantities of the cation and anion, a 1:1 equimolar ratio minimizes the impurities obtained by the reaction. The first and second aqueous solutions are mixed and stirred at a temperature that optimizes the separation of the desired product phase as either an oil or a solid on the bottom of the flask. In one embodiment, the aqueous solutions are mixed and stirred at room temperature, however the optimal temperature may be higher or lower based on the conditions necessary to achieve optimal product separation. The water layer is separated, and the product is washed several times with deionized water to remove chloride or bromide impurities. An additional base wash may help to remove acidic impurities. The product is then diluted with an appropriate organic solvent (chloroform, methylene chloride, etc.) and dried over anhydrous magnesium sulfate or other preferred drying agent. The appropriate organic solvent is one that is miscible with the ionic liquid and that can be dried. The drying agent is removed by suction filtration and the organic solvent is removed in vacuo. High vacuum is applied for several hours or until residual water is removed. The final product is usually in the form of a liquid.

Other ionic liquids suitable for use herein may be made as follows: A third solution is made by dissolving a known amount of the halide salt of the cation in an appropriate solvent. This may involve heating to ensure total dissolution. Preferably the solvent is one in which the cation and anion are miscible, and in which the salts formed by the reaction are minimally miscible; in addition, the appropriate solvent is preferably one that has a relatively low boiling point such that the solvent can be easily removed after the reaction. Appropriate solvents include, but are not limited to, high purity dry acetone, alcohols such as methanol and ethanol, and acetonitrile. A fourth solution is made by dissolving an equimolar amount (relative to the cation) of the salt (generally potassium or sodium) of the anion in an appropriate solvent, typically the same as that used for the cation. This may also involve heating to ensure total dissolution. The third and fourth solutions are mixed and stirred under conditions that result in approximately complete precipitation of the halide salt byproduct (generally potassium halide or sodium halide); in one embodiment of the invention, the solutions are mixed and stirred at approximately room temperature for about 4-12 hours. The halide salt is removed by suction filtration through an acetone/celite pad, and color can be reduced through the use of decolorizing carbon as is known to those skilled in the art. The solvent is removed in vacuo and then high vacuum is applied for several hours or until residual water is removed. The final product is usually in the form of a liquid.

The physical and chemical properties of ionic liquids will show some variation according to the identity of the cation and/or anion. For example, increasing the chain length of one or more alkyl chains of the cation will affect properties such as the melting point, hydrophilicity/lipophilicity, density and solvation strength of the ionic liquid. Choice of the anion can affect, for example, the melting point, the water solubility and the acidity and coordination properties of the composition. Effects of choice of cation and anion on the physical and chemical properties of ionic liquids are reviewed by Wasserscheid and Keim [Angew. Chem. Int. Ed. (2000) 39:3772-3789] and Sheldon [Chem. Commun. (2001) 2399-2407].

An ionic liquid may be present in the reaction mixture in an amount of about 0.1% or more, or about 2% or more, and yet in an amount of about 25% or less, or about 20% or less, by weight relative to the weight of the C₄ to C₈ alcohol present therein.

A catalyst suitable for use in a process hereof is a substance that increases the rate of approach to equilibrium of the reaction without itself being substantially consumed in the reaction. In preferred embodiments, the catalyst is a homogeneous catalyst in the sense that the catalyst and reactants occur in the same phase, which is uniform, and the catalyst is molecularly dispersed with the reactants in that phase.

In one embodiment, suitable acids for use herein as a homogeneous catalyst are those having a pKa of less than about 4; in another embodiment, suitable acids for use herein as a homogeneous catalyst are those having a pKa of less than about 2.

In one embodiment, a homogeneous acid catalyst suitable for use herein may be selected from the group consisting of inorganic acids, organic sulfonic acids, heteropolyacids, fluoroalkyl sulfonic acids, metal sulfonates, metal trifluoroacetates, compounds thereof and combinations thereof. In yet another embodiment, the homogeneous acid catalyst may be selected from the group consisting of sulfuric acid, fluorosulfonic acid, phosphorous acid, p-toluenesulfonic acid, benzenesulfonic acid, phosphotungstic acid, phosphomolybdic acid, trifluoromethanesulfonic acid, nonafluorobutanesulfonic acid, 1,1,2,2-tetrafluoroethanesulfonic acid, 1,1,2,3,3,3-hexafluoropropanesulfonic acid, bismuth triflate, yttrium triflate, ytterbium triflate, neodymium triflate, lanthanum triflate, scandium triflate, and zirconium triflate.

A catalyst may be present in the reaction mixture in an amount of about 0.1% or more, or about 1% or more, and yet in an amount of about 20% or less, or about 10% or less, or about 5% or less, by weight relative to the weight of the C₄ to C₈ alcohol present therein.

The reaction may be carried out at a temperature of from about 50 degrees C. to about 300 degrees C. In one embodiment, the temperature is from about 100 degrees C. to about 250 degrees C. The reaction may be carried out at a pressure of from about atmospheric pressure (about 0.1 MPa) to about 20.7 MPa. In a more specific embodiment, the pressure is from about 0.1 MPa to about 3.45 MPa. The reaction may be carried out under an inert atmosphere, for which inert gases such as nitrogen, argon and helium are suitable.

In one embodiment, the reaction is carried out in the liquid phase. In an alternative embodiment, the reaction is carried out at an elevated temperature and/or pressure such that the product dialkyl ethers are present in a vapor phase. Such vapor phase dialkyl ethers can be condensed to a liquid by reducing the temperature and/or pressure. The reduction in temperature and/or pressure can occur in the reaction vessel itself, or alternatively the vapor phase can be collected in a separate vessel, where the vapor phase is then condensed to a liquid phase.

The time for the reaction will depend on many factors, such as the reactants, reaction conditions and reactor, and may be adjusted to achieve high yields of dialkyl ethers. The reaction can be carried out in batch mode, or in continuous mode.

An advantage to the use of an ionic liquid in this reaction is that, as a result of the formation of the dialkyl ether product, the dialkyl ether product resides in a first phase (a “dialkyl ether phase”) of the reaction mixture that is separate from a second phase (an “ionic liquid phase”) in which the ionic liquid and catalyst reside. Thus the dialkyl ether product or products (in the dialkyl ether phase) is/are easily recoverable from the acid catalyst (in the ionic liquid phase) by, for example, decantation.

In another embodiment, the separated ionic liquid phase may be recycled for addition again to the reaction mixture. The conversion of one or more n-alcohols to one or more dialkyl ethers results in the formation of water. Therefore, where it is desired to recycle the ionic liquid contained in the ionic liquid phase, it may be necessary to treat the ionic liquid phase to remove water. One common treatment method for the removal of water is the use of distillation. Ionic liquids have negligible vapor pressure, and the catalysts useful in this invention generally have boiling points above that of water; therefore it is generally possible when distilling the ionic liquid phase to remove water from the top of a distillation column, whereas an ionic liquid and a catalyst would be removed from the bottom of the column. Methods of distillation applicable to the separation of water from an ionic liquid are further discussed in Section 13, “Distillation” of Perry's Chemical Engineers' Handbook, 7^{th} Ed. (McGraw-Hill, 1997). In further steps, catalyst residue may be separated from an ionic liquid by filtration or centrifugation, or catalyst residue may be returned to the reaction mixture along with the ionic liquid.

The separated and/or recovered dialkyl ether phase can optionally be further purified and used as such.

In various other embodiments of this invention, an ionic liquid formed by selecting any of the individual cations described or disclosed herein, and by selecting any of the individual anions described or disclosed herein, may be used in a reaction mixture to prepare a dialkyl ether. Correspondingly, in yet other embodiments, a subgroup of ionic liquids formed by selecting (i) a subgroup of any size of cations, taken from the total group of cations described and disclosed herein in all the various different combinations of the individual members of that total group, and (ii) a subgroup of any size of anions, taken from the total group of anions described and disclosed herein in all the various different combinations of the individual members of that total group, may be used in a reaction mixture to prepare a dialkyl ether. In forming an ionic liquid, or a subgroup of ionic liquids, by making selections as aforesaid, the ionic liquid or subgroup will be used in the absence of the members of the group of cations and/or anions that are omitted from the total group thereof to make the selection, and, if desirable, the selection may thus be made in terms of the members of the total group that are omitted from use rather than the members of the group that are included for use.

Each of the formulae shown herein describes each and all of the separate, individual compounds that can be assembled in that formula by (1) selection from within the prescribed range for one of the variable radicals, substituents or numerical coefficients while all of the other variable radicals, substituents or numerical coefficients are held constant, and (2) performing in turn the same selection from within the prescribed range for each of the other variable radicals, substituents or numerical coefficients with the others being held constant. In addition to a selection made within the prescribed range for any of the variable radicals, substituents or numerical coefficients of only one of the members of the group described by the range, a plurality of compounds may be described by selecting more than one but less than all of the members of the whole group of radicals, substituents or numerical coefficients. When the selection made within the prescribed range for any of the variable radicals, substituents or numerical coefficients is a subgroup containing (i) only one of the members of the whole group described by the range, or (ii) more than one but less than all of the members of the whole group, the selected member(s) are selected by omitting those member(s) of the whole group that are not selected to form the subgroup. The compound, or plurality of compounds, may in such event be characterized by a definition of one or more of the variable radicals, substituents or numerical coefficients that refers to the whole group of the prescribed range for that variable but where the member(s) omitted to form the subgroup are absent from the whole group.

The advantageous attributes and effects of the processes hereof may be seen in a series of examples (Examples 1˜2), as described below. The embodiments of these processes on which the examples are based are representative only, and the selection of those embodiments to illustrate the invention does not indicate that conditions, arrangements, approaches, regimes, reactants, techniques or protocols not described in these examples are not suitable for practicing these processes, or that subject matter not described in these examples is excluded from the scope of the appended claims and equivalents thereof.

### General Materials and Methods

The following abbreviations are used:

Nuclear magnetic resonance is abbreviated NMR; gas chromatography is abbreviated GC; gas chromatography-mass spectrometry is abbreviated GC-MS; thin layer chromatography is abbreviated TLC; thermogravimetric analysis (using a Universal V3.9A TA instrument analyser (TA Instruments, Inc., Newcastle, Del.)) is abbreviated TGA. Centigrade is abbreviated C, mega Pascal is abbreviated MPa, gram is abbreviated g, kilogram is abbreviated Kg, milliliter(s) is abbreviated ml(s), hour is abbreviated hr or h; weight percent is abbreviated wt %; milliequivalents is abbreviated meq; melting point is abbreviated Mp; differential scanning calorimetry is abbreviated DSC.

1-Butyl-2,3-dimethylimidazolium chloride, 1-hexyl-3-methylimidazolium chloride, 1-dodecyl-3-methylimidazolium chloride, 1-hexadecyl-3-methyl imidazolium chloride, 1-octadecyl-3-methylimidazolium chloride, imidazole, tetrahydrofuran, iodopropane, acetonitrile, iodoperfluorohexane, toluene, 1-butanol, oleum (20% SO₃), sodium sulfite (Na₂SO₃, 98%), and acetone were obtained from Acros (Hampton, N.H.). Potassium metabisulfite (K₂S₂O₅, 99%), was obtained from Mallinckrodt Laboratory Chemicals (Phillipsburg, N.J.). Potassium sulfite hydrate (KHSO₃.xH₂O, 95%), sodium bisulfite (NaHSO₃), sodium carbonate, magnesium sulfate, phosphotungstic acid, ethyl ether, 1,1,1,2,2,3,3,4,4,5,5,6,6-tridecafluoro-8-iodooctane, trioctyl phosphine and 1-ethyl-3-methylimidazolium chloride (98%) were obtained from Aldrich (St. Louis, Mo.). Sulfuric acid and methylene chloride were obtained from EMD Chemicals, Inc. (Gibbstown, N.J.). Perfluoro(ethylvinyl ether), perfluoro(methylvinyl ether), hexafluoropropene and tetrafluoroethylene were obtained from DuPont Fluoroproducts (Wilmington, Del.). 1-Butyl-methylimidazolium chloride was obtained from Fluka (Sigma-Aldrich, St. Louis, Mo.). Tetra-n-butylphosphonium bromide and tetradecyl(tri-n-hexyl)phosphonium chloride were obtained from Cytec (Canada Inc., Niagara Falls, Ontario, Canada). 1,1,2,2-Tetrafluoro-2-(pentafluoroethoxy)sulfonate was obtained from SynQuest Laboratories, Inc. (Alachua, Fla.).

### Preparation of Anions

### (A) Synthesis of potassium 1,1,2,2-tetrafluoroethanesulfonate (TFES-K) ([HCF₂CF₂SO₃]⁻)

A 1-gallon Hastelloy® C276 reaction vessel was charged with a solution of potassium sulfite hydrate (176 g, 1.0 mol), potassium metabisulfite (610 g, 2.8 mol) and deionized water (2000 ml). The pH of this solution was 5.8. The vessel was cooled to 18 degrees C., evacuated to 0.10 MPa, and purged with nitrogen. The evacuate/purge cycle was repeated two more times. To the vessel was then added tetrafluoroethylene (TFE, 66 g), and it was heated to 100 degrees C. at which time the inside pressure was 1.14 MPa. The reaction temperature was increased to 125 degrees C. and kept there for 3 hr. As the TFE pressure decreased due to the reaction, more TFE was added in small aliquots (20-g each) to maintain operating pressure roughly between 1.14 and 1.48 MPa. Once 500 g (5.0 mol) of TFE had been fed after the initial 66 g precharge, the vessel was vented and cooled to 25 degrees C. The pH of the clear light yellow reaction solution was 10-11. This solution was buffered to pH 7 through the addition of potassium metabisulfite (16 g).

The water was removed in vacuo on a rotary evaporator to produce a wet solid. The solid was then placed in a freeze dryer (Virtis Freezemobile 35xl; Gardiner, N.Y.) for 72 hr to reduce the water content to approximately 1.5 wt % (1387 g crude material). The theoretical mass of total solids was 1351 g. The mass balance was very close to ideal and the isolated solid had slightly higher mass due to moisture. This added freeze drying step had the advantage of producing a free-flowing white powder whereas treatment in a vacuum oven resulted in a soapy solid cake that was very difficult to remove and had to be chipped and broken out of the flask.

The crude TFES-K can be further purified and isolated by extraction with reagent grade acetone, filtration, and drying.

¹⁹F NMR (D₂O) δ −122.0 (dt, J_{FH}=6 Hz, J_{FF}=6 Hz, 2F); −136.1 (dt, J_{FH}=53 Hz, 2F).

¹H NMR (D₂O) δ 6.4 (tt, J_{FH}=53 Hz, J_{FH}=6 Hz, 1H).

% Water by Karl-Fisher titration: 580 ppm.

Analytical calculation for C₂HO₂F₄SK: C, 10.9; H, 0.5; N, 0.0. Experimental results: C, 11.1; H, 0.7; N, 0.2.

Mp (DSC): 242 degrees C.

TGA (air): 10% wt. loss @ 367 degrees C., 50% wt. loss @ 375 degrees C.

TGA (N₂): 10% wt. loss @ 363 degrees C., 50% wt. loss @ 375 degrees C.

### (B) Synthesis of potassium-1,1,2-trifluoro-2-(perfluoroethoxy)ethanesulfonate (TPES-K)

A 1-gallon Hastelloy® C276 reaction vessel was charged with a solution of potassium sulfite hydrate (88 g, 0.56 mol), potassium metabisulfite (340 g, 1.53 mol) and deionized water (2000 ml). The vessel was cooled to 7 degrees C., evacuated to 0.05 MPa, and purged with nitrogen. The evacuate/purge cycle was repeated two more times. To the vessel was then added perfluoro(ethylvinyl ether) (PEVE, 600 g, 2.78 mol), and it was heated to 125 degrees C. at which time the inside pressure was 2.31 MPa. The reaction temperature was maintained at 125 degrees C. for 10 hr. The pressure dropped to 0.26 MPa at which point the vessel was vented and cooled to 25 degrees C. The crude reaction product was a white crystalline precipitate with a colorless aqueous layer (pH=7) above it.

The ¹⁹F NMR spectrum of the white solid showed pure desired product, while the spectrum of the aqueous layer showed a small but detectable amount of a fluorinated impurity. The desired isomer is less soluble in water so it precipitated in isomerically pure form. The product slurry was suction filtered through a fritted glass funnel, and the wet cake was dried in a vacuum oven (60 degrees C., 0.01 MPa) for 48 hr. The product was obtained as off-white crystals (904 g, 97% yield).

¹⁹F NMR (D₂O) δ −86.5 (s, 3F); −89.2, −91.3 (subsplit ABq, J_{FF}=147 Hz, 2F); −119.3, −121.2 (subsplit ABq, J_{FF}=258 Hz, 2F); −144.3 (dm, J_{FH}=53 Hz, 1F).

¹H NMR (D₂O) δ 6.7 (dm, J_{FH}=53 Hz, 1H).

Mp (DSC) 263 degrees C.

Analytical calculation for C₄HO₄F₈SK: C, 14.3; H, 0.3.

Experimental results: C, 14.1; H, 0.3.

TGA (air): 10% wt. loss @ 359 degrees C., 50% wt. loss @ 367 degrees C.

TGA (N₂): 10% wt. loss @ 362 degrees C., 50% wt. loss @ 374 degrees C.

### (C) Synthesis of potassium-1,1,2-trifluoro-2-(trifluoromethoxy)ethanesulfonate (TIES-K)

A 1-gallon Hastelloy® C276 reaction vessel was charged with a solution of potassium sulfite hydrate (114 g, 0.72 mol), potassium metabisulfite (440 g, 1.98 mol) and deionized water (2000 ml). The pH of this solution was 5.8. The vessel was cooled to −35 degrees C., evacuated to 0.08 MPa, and purged with nitrogen. The evacuate/purge cycle was repeated two more times. To the vessel was then added perfluoro(methylvinyl ether) (PMVE, 600 g, 3.61 mol) and it was heated to 125 degrees C. at which time the inside pressure was 3.29 MPa. The reaction temperature was maintained at 125 degrees C. for 6 hr. The pressure dropped to 0.27 MPa at which point the vessel was vented and cooled to 25 degrees C. Once cooled, a white crystalline precipitate of the desired product formed leaving a colorless clear aqueous solution above it (pH=7).

The ¹⁹F NMR spectrum of the white solid showed pure desired product, while the spectrum of the aqueous layer showed a small but detectable amount of a fluorinated impurity. The solution was suction filtered through a fritted glass funnel for 6 hr to remove most of the water. The wet cake was then dried in a vacuum oven at 0.01 MPa and 50 degrees C. for 48 hr. This gave 854 g (83% yield) of a white powder. The final product was isomerically pure (by ¹⁹F and ¹H NMR) since the undesired isomer remained in the water during filtration.

¹⁹F NMR (D₂O) δ −59.9 (d, J_{FH}=4 Hz, 3F); −119.6, −120.2 (subsplit ABq, J=260 Hz, 2F); −144.9 (dm, J_{FH}=53 Hz, 1F).

¹H NMR (D₂O) δ 6.6 (dm, J_{FH}=53 Hz, 1H).

% Water by Karl-Fisher titration: 71 ppm.

Analytical calculation for C₃HF₆SO₄K: C, 12.6; H, 0.4; N, 0.0. Experimental results: C, 12.6; H, 0.0; N, 0.1.

Mp (DSC) 257 degrees C.

TGA (air): 10% wt. loss @ 343 degrees C., 50% wt. loss @ 358 degrees C.

TGA (N₂): 10% wt. loss @ 341 degrees C., 50% wt. loss @ 357 degrees C.

### (D) Synthesis of sodium 1,1,2,3,3,3-hexafluoropropanesulfonate (HFPS-Na)

A 1-gallon Hastelloy® C reaction vessel was charged with a solution of anhydrous sodium sulfite (25 g, 0.20 mol), sodium bisulfite 73 g, (0.70 mol) and of deionized water (400 ml). The pH of this solution was 5.7. The vessel was cooled to 4 degrees C., evacuated to 0.08 MPa, and then charged with hexafluoropropene (HFP, 120 g, 0.8 mol, 0.43 MPa). The vessel was heated with agitation to 120 degrees C. and kept there for 3 hr. The pressure rose to a maximum of 1.83 MPa and then dropped down to 0.27 MPa within 30 minutes. At the end, the vessel was cooled and the remaining HFP was vented, and the reactor was purged with nitrogen. The final solution had a pH of 7.3.

The water was removed in vacuo on a rotary evaporator to produce a wet solid. The solid was then placed in a vacuum oven (0.02 MPa, 140 degrees C., 48 hr) to produce 219 g of white solid which contained approximately 1 wt % water. The theoretical mass of total solids was 217 g.

The crude HFPS-Na can be further purified and isolated by extraction with reagent grade acetone, filtration, and drying.

¹⁹F NMR (D₂O) δ −74.5 (m, 3F); −113.1, −120.4 (ABq, J=264 Hz, 2F); −211.6 (dm, 1F).

¹H NMR (D₂O) δ 5.8 (dm, J_{FH}=43 Hz, 1H).

Mp (DSC) 126 degrees C.

TGA (air): 10% wt. loss @ 326 degrees C., 50% wt. loss @ 446 degrees C.

TGA (N₂): 10% wt. loss @ 322 degrees C., 50% wt. loss @ 449 degrees C.

### Preparation of Ionic Liquids

### (E) Synthesis of 1-butyl-2,3-dimethylimidazolium 1,1,2,2-tetrafluoroethanesulfonate (Cation, imidazolium; Anion, Formula I)

1-Butyl-2,3-dimethylimidazolium chloride (22.8 g, 0.121 moles) was mixed with reagent-grade acetone (250 ml) in a large round-bottomed flask and stirred vigorously. Potassium 1,1,2,2-tetrafluoroethanesulfonate (TFES-K, 26.6 g, 0.121 moles) was added to reagent grade acetone (250 ml) in a separate round-bottomed flask, and this solution was carefully added to the 1-butyl-2,3-dimethylimidazolium chloride solution. The large flask was lowered into an oil bath and heated at 60 degrees C. under reflux for 10 hours. The reaction mixture was then filtered using a large frit glass funnel to remove the white KCl precipitate formed, and the filtrate was placed on a rotary evaporator for 4 hours to remove the acetone. The product was isolated and dried under vacuum at 150 degrees C. for 2 days.

¹H NMR (DMSO-d₆): δ 0.9 (t, 3H); 1.3 (m, 2H); 1.7 (m, 2H); 2.6 (s, 3H); 3.8 (s, 3H); 4.1 (t, 2H); 6.4 (tt, 1H); 7.58 (s, 1H); 7.62 (s, 1H).

% Water by Karl-Fischer titration: 0.06%.

TGA (air): 10% wt. loss @ 375 degrees C., 50% wt. loss @ 415 degrees C.

TGA (N₂): 10% wt. loss @ 395 degrees C., 50% wt. loss @ 425 degrees C.

The reaction scheme is shown below:

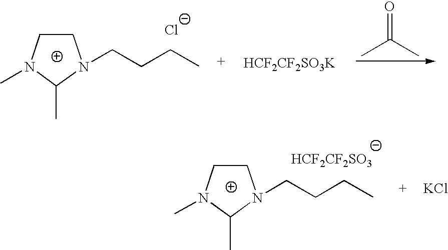

### (F) Synthesis of 1-butyl-3-methylimidazolium 1,1,2,2-tetrafluoroethanesulfonate (Bmim-TFES)

1-Butyl-3-methylimidazolium chloride (60.0 g) and high purity dry acetone (>99.5%, 300 ml) were combined in a 1 liter flask and warmed to reflux with magnetic stirring until the solid completely dissolved. At room temperature in a separate 1 liter flask, potassium-1,1,2,2-tetrafluoroethanesulfonte (TFES-K, 75.6 g) was dissolved in high purity dry acetone (500 ml). These two solutions were combined at room temperature and allowed to stir magnetically for 2 hr under positive nitrogen pressure. The stirring was stopped and the KCl precipitate was allowed to settle, then removed by suction filtration through a fritted glass funnel with a celite pad. The acetone was removed in vacuo to give a yellow oil. The oil was further purified by diluting with high purity acetone (100 ml) and stirring with decolorizing carbon (5 g). The mixture was again suction filtered and the acetone removed in vacuo to give a colorless oil. This was further dried at 4 Pa and 25 degrees C. for 6 hr to provide 83.6 g of product.

¹⁹F NMR (DMSO-d₆) δ −124.7 (dt, J=6 Hz, J=8 Hz, 2F); −136.8 (dt, J=53 Hz, 2F).

¹H NMR (DMSO-d₆) δ 0.9 (t, J=7.4 Hz, 3H); 1.3 (m, 2H); 1.8 (m, 2H); 3.9 (s, 3H); 4.2 (t, J=7 Hz, 2H); 6.3 (dt, J=53 Hz, J=6 Hz, 1H); 7.4 (s, 1H); 7.5 (s, 1H); 8.7 (s, 1H).

% Water by Karl-Fisher titration: 0.14%.

Analytical calculation for C₉H₁₂F₆N₂O₃S: C, 37.6; H, 4.7; N, 8.8. Experimental Results: C, 37.6; H, 4.6; N, 8.7.

TGA (air): 10% wt. loss @ 380 degrees C., 50% wt. loss @ 420 degrees C.

TGA (N₂): 10% wt. loss @ 375 degrees C., 50% wt. loss @ 422 degrees C.

### (G) Synthesis of 1-ethyl-3-methylimidazolium 1,1,2,2-tetrafluoroethanesulfonate (Emim-TFES)

To a 500 ml round bottom flask was added 1-ethyl-3-methylimidazolium chloride (Emim-Cl, 98%, 61.0 g) and reagent grade acetone (500 ml). The mixture was gently warmed (50 degrees C.) until almost all of the Emim-Cl dissolved. To a separate 500 ml flask was added potassium 1,1,2,2-tetrafluoroethanesulfonate (TFES-K, 90.2 g) along with reagent grade acetone (350 ml). This second mixture was stirred magnetically at 24 degrees C. until all of the TFES-K dissolved.

These solutions were combined in a 1 liter flask producing a milky white suspension. The mixture was stirred at 24 degrees C. for 24 hrs. The KCl precipitate was then allowed to settle leaving a clear green solution above it.

The reaction mixture was filtered once through a celite/acetone pad and again through a fritted glass funnel to remove the KCl. The acetone was removed in vacuo first on a rotovap and then on a high vacuum line (4 Pa, 25 degrees C.) for 2 hr. The product was a viscous light yellow oil (76.0 g, 64% yield).

¹⁹F NMR (DMSO-d₆) δ −124.7 (dt, J_{FH}=6 Hz, J_{FF}=6 Hz, 2F); −138.4 (dt, J_{FH}=53 Hz, 2F).

¹H NMR (DMSO-d₆) δ 1.3 (t, J=7.3 Hz, 3H); 3.7 (s, 3H); 4.0 (q, J=7.3 Hz, 2H); 6.1 (tt, J_{FH}=53 Hz, J_{FH}=6 Hz, 1H); 7.2 (s, 1H); 7.3 (s, 1H); 8.5 (s, 1H).

% Water by Karl-Fisher titration: 0.18%.

Analytical calculation for C₈H₁₂N₂O₃F₄S: C, 32.9; H, 4.1; N, 9.6. Found: C, 33.3; H, 3.7; N, 9.6.

Mp 45-46 degrees C.

TGA (air): 10% wt. loss @ 379 degrees C., 50% wt. loss @ 420 degrees C.

TGA (N₂): 10% wt. loss @ 378 degrees C., 50% wt. loss @ 418 degrees C.

The reaction scheme is shown below:

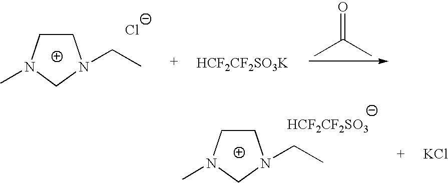

### (H) Synthesis of 1-ethyl-3-methylimidazolium 1,1,2,3,3,3-hexafluoropropanesulfonate (Emim-HFPS)

To a 11 round bottom flask was added 1-ethyl-3-methylimidazolium chloride (Emim-C1, 98%, 50.5 g) and reagent grade acetone (400 ml). The mixture was gently warmed (50 degrees C.) until almost all of the Emim-Cl dissolved. To a separate 500 ml flask was added potassium 1,1,2,3,3,3-hexafluoropropanesulfonate (HFPS-K, 92.2 g) along with reagent grade acetone (300 ml). This second mixture was stirred magnetically at room temperature until all of the HFPS-K dissolved.

These solutions were combined and stirred under positive N₂ pressure at 26 degrees C. for 12 hr producing a milky white suspension. The KCl precipitate was allowed to settle overnight leaving a clear yellow solution above it.

The reaction mixture was filtered once through a celite/acetone pad and again through a fritted glass funnel. The acetone was removed in vacuo first on a rotovap and then on a high vacuum line (4 Pa, 25 degrees C.) for 2 hr. The product was a viscious light yellow oil (103.8 g, 89% yield).

¹⁹F NMR (DMSO-d₆) δ −73.8 (s, 3F); −114.5, −121.0 (ABq, J=258 Hz, 2F); −210.6 (m, 1F, J_{HF}=41.5 Hz).

¹H NMR (DMSO-d₆) δ 1.4 (t, J=7.3 Hz, 3H); 3.9 (s, 3H); 4.2 (q, J=7.3 Hz, 2H,); 5.8 (m, J_{HF}=41.5 Hz, 1H,); 7.7 (s, 1H); 7.8 (s, 1H); 9.1 (s, 1H).

% Water by Karl-Fisher titration: 0.12%.

Analytical calculation for C₉H₁₂N₂O₃F₆S: C, 31.5; H, 3.5; N, 8.2. Experimental Results: C, 30.9; H, 3.3; N, 7.8.

TGA (air): 10% wt. loss @ 342 degrees C., 50% wt. loss @ 373 degrees C.

TGA (N₂): 10% wt. loss @ 341 degrees C., 50% wt. loss @ 374 degrees C.

The reaction scheme is shown below:

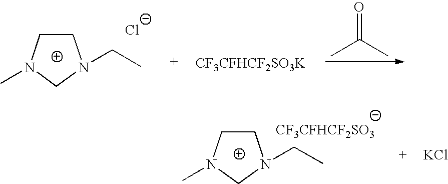

### (I) Synthesis of 1-hexyl-3-methylimidazolium 1,1,2,2-tetrafluoroethanesulfonate

1-Hexyl-3-methylimidazolium chloride (10 g, 0.0493 moles) was mixed with reagent-grade acetone (100 ml) in a large round-bottomed flask and stirred vigorously under a nitrogen blanket. Potassium 1,1,2,2-tetrafluoroethane sulfonate (TFES-K, 10 g, 0.0455 moles) was added to reagent grade acetone (100 ml) in a separate round-bottomed flask, and this solution was carefully added to the 1-hexyl-3-methylimidazolium chloride/acetone mixture. The mixture was left to stir overnight. The reaction mixture was then filtered using a large frit glass funnel to remove the white KCl precipitate formed, and the filtrate was placed on a rotary evaporator for 4 hours to remove the acetone.

Appearance: pale yellow, viscous liquid at room temperature.

¹H NMR (DMSO-d₆): δ 0.9 (t, 3H); 1.3 (m, 6H); 1.8 (m, 2H); 3.9 (s, 3H); 4.2 (t, 2H); 6.4 (tt, 1H); 7.7 (s, 1H); 7.8 (s, 1H); 9.1 (s, 1H).

% Water by Karl-Fischer titration: 0.03%

TGA (air): 10% wt. loss @ 365 degrees C., 50% wt. loss @ 410 degrees C.

TGA (N₂): 10% wt. loss @ 370 degrees C., 50% wt. loss @ 415 degrees C.

The reaction scheme is shown below:

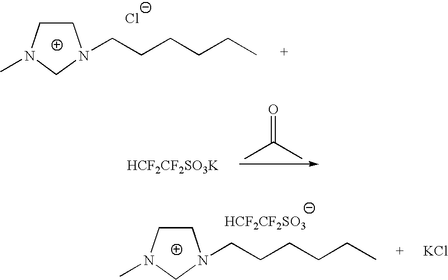

### (J) Synthesis of 1-dodecyl-3-methylimidazolium 1,1,2,2-tetrafluoroethanesulfonate

1-Dodecyl-3-methylimidazolium chloride (34.16 g, 0.119 moles) was partially dissolved in reagent-grade acetone (400 ml) in a large round-bottomed flask and stirred vigorously. Potassium 1,1,2,2-tetrafluoroethanesulfonate (TFES-K, 26.24 g, 0.119 moles) was added to reagent grade acetone (400 ml) in a separate round-bottomed flask, and this solution was carefully added to the 1-dodecyl-3-methylimidazolium chloride solution. The reaction mixture was heated at 60 degrees C. under reflux for approximately 16 hours. The reaction mixture was then filtered using a large frit glass funnel to remove the white KCl precipitate formed, and the filtrate was placed on a rotary evaporator for 4 hours to remove the acetone.

¹H NMR (CD₃CN): δ 0.9 (t, 3H); 1.3 (m. 18H); 1.8 (m, 2H); 3.9 (s, 3H); 4.2 (t, 2H); 6.4 (tt, 1H); 7.7 (s, 1H); 7.8 (s, 1H); 9.1 (s, 1H).

¹⁹F NMR (CD₃CN): δ −125.3 (m, 2F); −137 (dt, 2F).

% Water by Karl-Fischer titration: 0.24%

TGA (air): 10% wt. loss @ 370 degrees C., 50% wt. loss @ 410 degrees C.

TGA (N₂): 10% wt. loss @ 375 degrees C., 50% wt. loss @ 410 degrees C.

The reaction scheme is shown below:

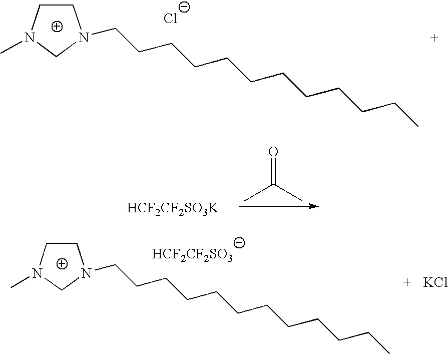

### (K) Synthesis of 1-hexadecyl-3-methylimidazolium 1,1,2,2-tetrafluoroethanesulfonate

1-Hexadecyl-3-methylimidazolium chloride (17.0 g, 0.0496 moles) was partially dissolved in reagent-grade acetone (100 ml) in a large round-bottomed flask and stirred vigorously. Potassium 1,1,2,2-tetrafluoroethanesulfonate (TFES-K, 10.9 g, 0.0495 moles) was added to reagent grade acetone (100 ml) in a separate round-bottomed flask, and this solution was carefully added to the 1-hexadecyl-3-methylimidazolium chloride solution. The reaction mixture was heated at 60 degrees C. under reflux for approximately 16 hours. The reaction mixture was then filtered using a large frit glass funnel to remove the white KCl precipitate formed, and the filtrate was placed on a rotary evaporator for 4 hours to remove the acetone.

Appearance: white solid at room temperature.

¹H NMR (CD₃CN): δ 0.9 (t, 3H); 1.3 (m, 26H); 1.9 (m, 2H); 3.9 (s, 3H); 4.2 (t, 2H); 6.3 (tt, 1H); 7.4 (s, 1H); 7.4 (s, 1H); 8.6 (s, 1H).

¹⁹F NMR (CD₃CN): δ −125.2 (m, 2F); −136.9 (dt, 2F).

% Water by Karl-Fischer titration: 200 ppm.

TGA (air): 10% wt. loss @ 360 degrees C., 50% wt. loss @ 395 degrees C.

TGA (N₂): 10% wt. loss @ 370 degrees C., 50% wt. loss @ 400 degrees C.

The reaction scheme is shown below:

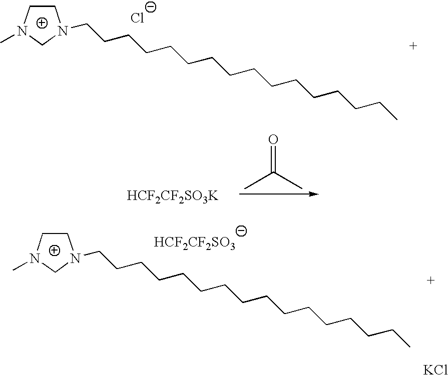

### (L) Synthesis of 1-octadecyl-3-methylimidazolium 1,1,2,2-tetrafluoroethanesulfonate

1-Octadecyl-3-methylimidazolium chloride (17.0 g, 0.0458 moles) was partially dissolved in reagent-grade acetone (200 ml) in a large round-bottomed flask and stirred vigorously. Potassium 1,1,2,2-tetrafluoroethanesulfonate (TFES-K, 10.1 g, 0.0459 moles), was added to reagent grade acetone (200 ml) in a separate round-bottomed flask, and this solution was carefully added to the 1-octadecyl-3-methylimidazolium chloride solution. The reaction mixture was heated at 60 degrees C. under reflux for approximately 16 hours. The reaction mixture was then filtered using a large frit glass funnel to remove the white KCl precipitate formed, and the filtrate was placed on a rotary evaporator for 4 hours to remove the acetone.

¹H NMR (CD₃CN): δ 0.9 (t, 3H); 1.3 (m, 30H); 1.9 (m, 2H); 3.9 (s, 3H); 4.1 (t, 2H); 6.3 (tt, 1H); 7.4 (s, 1H); 7.4 (s, 1H); 8.5 (s, 1H).

¹⁹F NMR (CD₃CN): δ −125.3 (m, 2F); −136.9 (dt, 2F).

% Water by Karl-Fischer titration: 0.03%.

TGA (air): 10% wt. loss @ 360 degrees C., 50% wt. loss @ 400 degrees C.

TGA (N₂): 10% wt. loss @ 365 degrees C., 50% wt. loss @ 405 degrees C.

The reaction scheme is shown below:

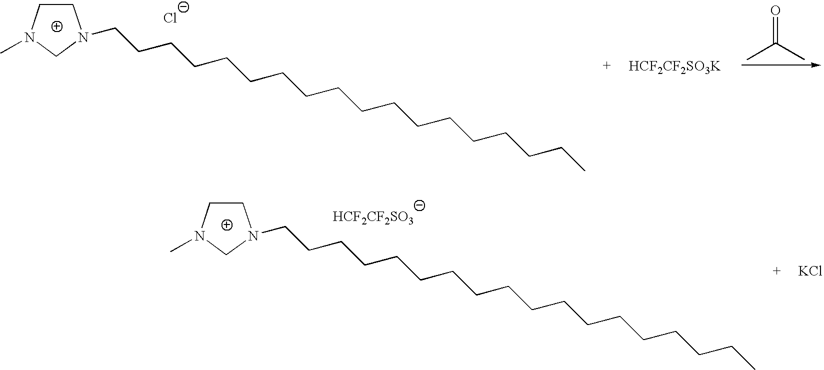

### (M) Synthesis of N-(1,1,2,2-tetrafluoroethyl)propylimidazole 1,1,2,2-tetrafluoroethanesulfonate

Imidazole (19.2 g) was added to of tetrahydrofuran (80 mls). A glass shaker tube reaction vessel was filled with the THF-containing imidazole solution. The vessel was cooled to 18° C., evacuated to 0.08 MPa, and purged with nitrogen. The evacuate/purge cycle was repeated two more times. Tetrafluoroethylene (TFE, 5 g) was then added to the vessel, and it was heated to 100 degrees C., at which time the inside pressure was about 0.72 MPa. As the TFE pressure decreased due to the reaction, more TFE was added in small aliquots (5 g each) to maintain operating pressure roughly between 0.34 MPa and 0.86 MPa. Once 40 g of TFE had been fed, the vessel was vented and cooled to 25 degrees C. The THF was then removed under vacuum and the product was vacuum distilled at 40 degrees C. to yield pure product as shown by ¹H and ¹⁹F NMR (yield 44 g). Iodopropane (16.99 g) was mixed with 1-(1,1,2,2-tetrafluoroethyl)imidazole (16.8 g) in dry acetonitrile (100 ml), and the mixture was refluxed for 3 days. The solvent was removed in vacuo, yielding a yellow waxy solid (yield 29 g). The product, 1-propyl-3-(1,1,2,2-tetrafluoroethyl)imidazolium iodide was confirmed by ¹H NMR (in d acetonitrile) [0.96 (t, 3H); 1.99 (m, 2H); 4.27 (t, 2H); 6.75 (t, 1H); 7.72 (d, 2H); 9.95 (s, 1H)].

Iodide (24 g) was then added to 60 ml of dry acetone, followed by 15.4 g of potassium 1,1,2,2-tetrafluoroethanesulfonate in 75 ml of dry acetone. The mixture was heated at 60 degrees C. overnight and a dense white precipitate was formed (potassium iodide). The mixture was cooled, filtered, and the solvent from the filtrate was removed using a rotary evaporator. Some further potassium iodide was removed under filtration. The product was further purified by adding 50 g of acetone, 1 g of charcoal, 1 g of celite and 1 g of silica gel. The mixture was stirred for 2 hours, filtered and the solvent removed. This yielded 15 g of a liquid, shown by NMR to be the desired product.

### (N) Synthesis of 1-butyl-3-methylimidazolium 1,1,2,3,3,3-hexafluoropropanesulfonate (Bmim-HFPS)

1-Butyl-3-methylimidazolium chloride (Bmim-Cl, 50.0 g) and high purity dry acetone (>99.5%, 500 ml) were combined in a 1 liter flask and warmed to reflux with magnetic stirring until the solid all dissolved. At room temperature in a separate 1 liter flask, potassium-1,1,2,3,3,3-hexafluoropropanesulfonte (HFPS-K) was dissolved in high purity dry acetone (550 ml). These two solutions were combined at room temperature and allowed to stir magnetically for 12 hr under positive nitrogen pressure. The stirring was stopped, and the KCl precipitate was allowed to settle. This solid was removed by suction filtration through a fritted glass funnel with a celite pad. The acetone was removed in vacuo to give a yellow oil. The oil was further purified by diluting with high purity acetone (100 ml) and stirring with decolorizing carbon (5 g). The mixture was suction filtered and the acetone removed in vacuo to give a colorless oil. This was further dried at 4 Pa and 25 degrees C. for 2 hr to provide 68.6 g of product.

¹⁹F NMR (DMSO-d₆) δ −73.8 (s, 3F); −114.5, −121.0 (ABq, J=258 Hz, 2F); −210.6 (m, J=42 Hz, 1F).

¹H NMR (DMSO-d₆) δ 0.9 (t, J=7.4 Hz, 3H); 1.3 (m, 2H); 1.8 (m, 2H); 3.9 (s, 3H); 4.2 (t, J=7 Hz, 2H); 5.8 (dm, J=42 Hz, 1H); 7.7 (s, 1H); 7.8 (s, 1H); 9.1 (s, 1H).

% Water by Karl-Fisher titration: 0.12%.

Analytical calculation for C₉H₁₂F₆N₂O₃S: C, 35.7; H, 4.4; N, 7.6. Experimental Results: C, 34.7; H, 3.8; N, 7.2.

TGA (air): 10% wt. loss @ 340 degrees C., 50% wt. loss @ 367 degrees C.

TGA (N₂): 10% wt. loss @ 335 degrees C., 50% wt. loss @ 361 degrees C.

Extractable chloride by ion chromatography: 27 ppm.

### (O) Synthesis of 1-butyl-3-methylimidazolium 1,1,2-trifluoro-2-(trifluoromethoxy)ethanesulfonate (Bmim-TTES)

1-Butyl-3-methylimidazolium chloride (Bmim-Cl, 10.0 g) and deionized water (15 ml) were combined at room temperature in a 200 ml flask. At room temperature in a separate 200 ml flask, potassium 1,1,2-trifluoro-2-(trifluoromethoxy)ethanesulfonate (TIES-K, 16.4 g) was dissolved in deionized water (90 ml). These two solutions were combined at room temperature and allowed to stir magnetically for 30 min. under positive nitrogen pressure to give a biphasic mixture with the desired ionic liquid as the bottom phase. The layers were separated, and the aqueous phase was extracted with 2×50 ml portions of methylene chloride. The combined organic layers were dried over magnesium sulfate and concentrated in vacuo. The colorless oil product was dried at for 4 hr at 5 Pa and 25 degrees C. to afford 15.0 g of product.

¹⁹F NMR (DMSO-d₆) δ −56.8 (d, J_{FH}=4 Hz, 3F); −119.5, −119.9 (subsplit ABq, J=260 Hz, 2F); −142.2 (dm, J_{FH}=53 Hz, 1F).

¹H NMR (DMSO-d₆) δ 0.9 (t, J=7.4 Hz, 3H); 1.3 (m, 2H); 1.8 (m, 2H); 3.9 (s, 3H); 4.2 (t, J=7.0 Hz, 2H); 6.5 (dt, J=53 Hz, J=7 Hz, 1H); 7.7 (s, 1H); 7.8 (s, 1H); 9.1 (s, 1H).

% Water by Karl-Fisher titration: 613 ppm.

Analytical calculation for C₁₁H₁₆F₆N₂O₄S: C, 34.2; H, 4.2; N, 7.3. Experimental Results: C, 34.0; H, 4.0; N, 7.1.

TGA (air): 10% wt. loss @ 328 degrees C., 50% wt. loss @ 354 degrees C.

TGA (N₂): 10% wt. loss @ 324 degrees C., 50% wt. loss @ 351 degrees C.

Extractable chloride by ion chromatography: <2 ppm.

### (P) Synthesis of 1-butyl-3-methylimidazolium 1,1,2-trifluoro-2-(perfluoroethoxy)ethanesulfonate (Bmim-TPES)

1-Butyl-3-methylimidazolium chloride (Bmim-Cl, 7.8 g) and dry acetone (150 ml) were combined at room temperature in a 500 ml flask. At room temperature in a separate 200 ml flask, potassium 1,1,2-trifluoro-2-(perfluoroethoxy)ethanesulfonate (TPES-K, 15.0 g) was dissolved in dry acetone (300 ml). These two solutions were combined and allowed to stir magnetically for 12 hr under positive nitrogen pressure. The KCl precipitate was then allowed to settle leaving a colorless solution above it. The reaction mixture was filtered once through a celite/acetone pad and again through a fritted glass funnel to remove the KCl. The acetone was removed in vacuo first on a rotovap and then on a high vacuum line (4 Pa, 25 degrees C.) for 2 hr. Residual KCl was still precipitating out of the solution, so methylene chloride (50 ml) was added to the crude product which was then washed with deionized water (2×50 ml). The solution was dried over magnesium sulfate, and the solvent was removed in vacuo to give the product as a viscous light yellow oil (12.0 g, 62% yield).

¹⁹F NMR (CD₃CN) δ+−85.8 (s, 3F); −87.9, −90.1 (subsplit ABq, J_{FF}=147 Hz, 2F); −120.6, −122.4 (subsplit ABq, J_{FF}=258 Hz, 2F); −142.2 (dm, J_{FH}=53 Hz, 1F).

¹H NMR (CD₃CN) δ 1.0 (t, J=7.4 Hz, 3H); 1.4 (m, 2H); 1.8 (m, 2H); 3.9 (s, 3H); 4.2 (t, J=7.0 Hz, 2H); 6.5 (dm, J=53 Hz, 1H); 7.4 (s, 1H); 7.5 (s, 1H); 8.6 (s, 1H).

% Water by Karl-Fisher titration: 0.461.

Analytical calculation for C₁₂H₁₆F₈N₂O₄S: C, 33.0; H, 3.7. Experimental Results: C, 32.0; H, 3.6.

TGA (air): 10% wt. loss @ 334 degrees C., 50% wt. loss @ 353 degrees C.

TGA (N₂): 10% wt. loss @ 330 degrees C., 50% wt. loss @ 365 degrees C.

### (Q) Synthesis of tetradecyl(tri-n-butyl)phosphonium 1,1,2,3,3,3-hexafluoropropanesulfonate ([4.4.4.14]P-HFPS)

To a 41 round bottomed flask was added the ionic liquid tetradecyl(tri-n-butyl)phosphonium chloride (Cyphos® IL 167, 345 g) and deionized water (1000 ml). The mixture was magnetically stirred until it was one phase. In a separate 2 liter flask, potassium 1,1,2,3,3,3-hexafluoropropanesulfonate (HFPS-K, 214.2 g) was dissolved in deionized water (1100 ml). These solutions were combined and stirred under positive N₂ pressure at 26 degrees C. for 1 hr producing a milky white oil. The oil slowly solidified (439 g) and was removed by suction filtration and then dissolved in chloroform (300 ml). The remaining aqueous layer (pH=2) was extracted once with chloroform (100 ml). The chloroform layers were combined and washed with an aqueous sodium carbonate solution (50 ml) to remove any acidic impurity. They were then dried over magnesium sulfate, suction filtered, and reduced in vacuo first on a rotovap and then on a high vacuum line (4 Pa, 100 degrees C.) for 16 hr to yield the final product as a white solid (380 g, 76% yield).

¹⁹F NMR (DMSO-d₆) δ −73.7 (s, 3F); −114.6, −120.9 (ABq, J=258 Hz, 2F); −210.5 (m, J_{HF}=41.5 Hz, 1F).

¹H NMR (DMSO-d₆) δ 0.8 (t, J=7.0 Hz, 3H); 0.9 (t, J=7.0 Hz, 9H); 1.3 (br s, 20H); 1.4 (m, 16H); 2.2 (m, 8H); 5.9 (m, J_{HF}=42 Hz, 1H).

% Water by Karl-Fisher titration: 895 ppm.

Analytical calculation for C₂₉H₅₇F₆O₃PS: C, 55.2; H, 9.1; N, 0.0. Experimental Results: C, 55.1; H, 8.8; N, 0.0.

TGA (air): 10% wt. loss @ 373 degrees C., 50% wt. loss @ 421 degrees C.

TGA (N₂): 10% wt. loss @ 383 degrees C., 50% wt. loss @ 436 degrees C.

### (R) Synthesis of Tetradecyl(tri-n-hexyl)phosphonium 1,1,2-trifluoro-2-(perfluoroethoxy)ethanesulfonate ([6.6.6.14]P-TPES)

To a 500 ml round bottomed flask was added acetone (Spectroscopic grade, 50 ml) and ionic liquid tetradecyl(tri-n-hexyl)phosphonium chloride (Cyphos® IL 101, 33.7 g). The mixture was magnetically stirred until it was one phase. In a separate 1 liter flask, potassium 1,1,2-trifluoro-2-(perfluoroethoxy)ethanesulfonate (TPES-K, 21.6 g) was dissolved in acetone (400 ml). These solutions were combined and stirred under positive N₂ pressure at 26 degrees C. for 12 hr producing a white precipitate of KCl. The precipitate was removed by suction filtration, and the acetone was removed in vacuo on a rotovap to produce the crude product as a cloudy oil (48 g). Chloroform (100 ml) was added, and the solution was washed once with deionized water (50 ml). It was then dried over magnesium sulfate and reduced in vacuo first on a rotovap and then on a high vacuum line (8 Pa, 24 degrees C.) for 8 hr to yield the final product as a slightly yellow oil (28 g, 56% yield).

¹⁹F NMR (DMSO-d₆) δ −86.1 (s, 3F); −88.4, −90.3 (subsplit ABq, J_{FF}=147 Hz, 2F); −121.4, −122.4 (subsplit ABq, J_{FF}=258 Hz, 2F); −143.0 (dm, J_{FH}=53 Hz, 1F).

¹H NMR (DMSO-d₆) δ 0.9 (m, 12H); 1.2 (m, 16H); 1.3 (m, 16H); 1.4 (m, 8H); 1.5 (m, 8H); 2.2 (m, 8H); 6.3 (dm, J_{FH}=54 Hz, 1H).

% Water by Karl-Fisher titration: 0.11.

Analytical calculation for C₃₆H₆₉F₈O₄PS: C, 55.4; H, 8.9; N, 0.0. Experimental Results: C, 55.2; H, 8.2; N, 0.1.

TGA (air): 10% wt. loss @ 311 degrees C., 50% wt. loss @ 339 degrees C.

TGA (N₂): 10% wt. loss @ 315 degrees C., 50% wt. loss @ 343 degrees C.

### (S) Synthesis of tetradecyl(tri-n-hexyl)phosphonium 1,1,2-trifluoro-2-(trifluoromethoxy)ethanesulfonate ([6.6.6.14]P-TTES)

To a 100 ml round bottomed flask was added acetone (Spectroscopic grade, 50 ml) and ionic liquid tetradecyl(tri-n-hexyl)phosphonium chloride (Cyphos®IL 101, 20.2 g). The mixture was magnetically stirred until it became one phase. In a separate 100 ml flask, potassium 1,1,2-trifluoro-2-(trifluoromethoxy)ethanesulfonate (TIES-K, 11.2 g) was dissolved in acetone (100 ml). These solutions were combined and stirred under positive N₂ pressure at 26 degrees C. for 12 hr producing a white precipitate of KCl.

The precipitate was removed by suction filtration, and the acetone was removed in vacuo on a rotovap to produce the crude product as a cloudy oil. The product was diluted with ethyl ether (100 ml) and then washed once with deionized water (50 ml), twice with an aqueous sodium carbonate solution (50 ml) to remove any acidic impurity, and twice more with deionized water (50 ml). The ether solution was then dried over magnesium sulfate and reduced in vacuo first on a rotovap and then on a high vacuum line (4 Pa, 24 degrees C.) for 8 hr to yield the final product as an oil (19.0 g, 69% yield).

¹⁹F NMR (CD₂Cl₂) δ −60.2 (d, J_{FH}=4 Hz, 3F); −120.8, −125.1 (subsplit ABq, J=260 Hz, 2F); −143.7 (dm, J_{FH}=53 Hz, 1F).

¹H NMR (CD₂Cl₂) δ 0.9 (m, 12H); 1.2 (m, 16H); 1.3 (m, 16H); 1.4 (m, 8H); 1.5 (m, 8H); 2.2 (m, 8H); 6.3 (dm, J_{FH}=54 Hz, 1H).

% Water by Karl-Fisher titration: 412 ppm.

Analytical calculation for C₃₅H₆₉F₆O₄PS: C, 57.5; H, 9.5; N, 0.0. Experimental results: C, 57.8; H, 9.3; N, 0.0.

TGA (air): 10% wt. loss @ 331 degrees C., 50% wt. loss @ 359 degrees C.

TGA (N₂): 10% wt. loss @ 328 degrees C., 50% wt. loss @ 360 degrees C.

### (T) Synthesis of 1-ethyl-3-methylimidazolium 1,1,2,2-tetrafluoro-2-(pentafluoroethoxy)sulfonate (Emim-TPENTAS)

To a 500 ml round bottomed flask was added 1-ethyl-3-methylimidazolium chloride (Emim-Cl, 98%, 18.0 g) and reagent grade acetone (150 ml). The mixture was gently warmed (50 degrees C.) until all of the Emim-Cl dissolved. In a separate 500 ml flask, potassium 1,1,2,2-tetrafluoro-2-(pentafluoroethoxy)sulfonate (TPENTAS-K, 43.7 g) was dissolved in reagent grade acetone (450 ml).

These solutions were combined in a 1 liter flask producing a white precipitate (KCl). The mixture was stirred at 24 degrees C. for 8 hr. The KCl precipitate was then allowed to settle leaving a clear yellow solution above it. The KCl was removed by filtration through a celite/acetone pad. The acetone was removed in vacuo to give a yellow oil which was then diluted with chloroform (100 ml). The chloroform was washed three times with deionized water (50 ml), dried over magnesium sulfate, filtered, and reduced in vacuo first on a rotovap and then on a high vacuum line (4 Pa, 25 degrees C.) for 8 hr. The product was a light yellow oil (22.5 g).

¹⁹F NMR (DMSO-d₆) δ −82.9 (m, 2F); −87.3 (s, 3F); −89.0 (m, 2F); −118.9 (s, 2F).

¹H NMR (DMSO-d₆) δ 1.5 (t, J=7.3 Hz, 3H); 3.9 (s, 3H); 4.2 (q, J=7.3 Hz, 2H); 7.7 (s, 1H); 7.8 (s, 1H); 9.1 (s, 1H).

% Water by Karl-Fisher titration: 0.17%.

Analytical calculation for C₁₀H₁₁N₂O₄F₉S: C, 28.2; H, 2.6; N, 6.6. Experimental results: C, 28.1: H, 2.9; N, 6.6.

TGA (air): 10% wt. loss @ 351 degrees C., 50% wt. loss @ 401 degrees C.

TGA (N₂): 10% wt. loss @ 349 degrees C., 50% wt. loss @ 406 degrees C.

### (U) Synthesis of tetrabutylphosphonium 1,1,2-trifluoro-2-(perfluoroethoxy)ethanesulfonate (TBP-TPES)

To a 200 ml round bottomed flask was added deionized water (100 ml) and tetra-n-butylphosphonium bromide (Cytec Canada Inc., 20.2 g). The mixture was magnetically stirred until the solid all dissolved. In a separate 300 ml flask, potassium 1,1,2-trifluoro-2-(perfluoroethoxy)ethanesulfonate (TPES-K, 20.0 g) was dissolved in deionized water (400 ml) heated to 70 degrees C. These solutions were combined and stirred under positive N₂ pressure at 26 degrees C. for 2 hr producing a lower oily layer. The product oil layer was separated and diluted with chloroform (30 ml), then washed once with an aqueous sodium carbonate solution (4 ml) to remove any acidic impurity, and three times with deionized water (20 ml). It was then dried over magnesium sulfate and reduced in vacuo first on a rotovap and then on a high vacuum line (8 Pa, 24 degrees C.) for 2 hr to yield the final product as a colorless oil (28.1 g, 85% yield).

¹⁹F NMR (CD₂Cl₂) δ −86.4 (s, 3F); −89.0, −90.8 (subsplit ABq, J_{FF}=147 Hz, 2F); −119.2, −125.8 (subsplit ABq, J_{FF}=254 Hz, 2F); −141.7 (dm, J_{FH}=53 Hz, 1F).

¹H NMR (CD₂Cl₂) δ 1.0 (t, J=7.3 Hz, 12H); 1.5 (m, 16H); 2.2 (m, 8H); 6.3 (dm, J_{FH}=54 Hz, 1H).

% Water by Karl-Fisher titration: 0.29.

Analytical calculation for C₂₀H₃₇F₈O₄PS: C, 43.2; H, 6.7; N, 0.0. Experimental results: C, 42.0; H, 6.9; N, 0.1.

Extractable bromide by ion chromatography: 21 ppm.

### (V) Synthesis of (3,3,4,4,5,5,6,6,7,7,8,8,8-tridecafluorooctyl)-trioctylphosphonium 1,1,2,2-tetrafluoroethanesulfonate

Trioctyl phosphine (31 g) was partially dissolved in reagent-grade acetonitrile (250 ml) in a large round-bottomed flask and stirred vigorously. 1,1,1,2,2,3,3,4,4,5,5,6,6-Tridecafluoro-8-iodooctane (44.2 g) was added, and the mixture was heated under reflux at 110 degrees C. for 24 hours. The solvent was removed under vacuum giving (3,3,4,4,5,5,6,6,7,7,8,8,8-tridecafluorooctyl)-trioctylphosphonium iodide as a waxy solid (30.5 g). Potassium 1,1,2,2-tetrafluoroethanesulfonate (TFES-K, 13.9 g) was dissolved in reagent grade acetone (100 ml) in a separate round-bottomed flask, and to this was added (3,3,4,4,5,5,6,6,7,7,8,8,8-tridecafluorooctyl)-trioctylphosphonium iodide (60 g). The reaction mixture was heated at 60 degrees C. under reflux for approximately 16 hours. The reaction mixture was then filtered using a large frit glass funnel to remove the white KI precipitate formed, and the filtrate was placed on a rotary evaporator for 4 hours to remove the acetone. The liquid was left for 24 hours at room temperature and then filtered a second time (to remove KI) to yield the product (62 g) as shown by proton NMR.

### (W) Synthesis of 1-methyl-3-(3,3,4,4,5,5,6,6,7,7,8,8,8-tridecafluorooctyl)imidazolium 1,1,2,2-tetrafluoroethanesulfonate

1-Methylimidazole (4.32 g, 0.52 mol) was partially dissolved in reagent-grade toluene (50 ml) in a large round-bottomed flask and stirred vigorously. 1,1,1,2,2,3,3,4,4,5,5,6,6-Tridecafluoro-8-iodooctane (26 g, 0.053 mol) was added, and the mixture was heated under reflux at 110 degrees C. for 24 hours. The solvent was removed under vacuum giving 1-methyl-3-(3,3,4,4,5,5,6,6,7,7,8,8,8-tridecafluorooctyl)imidazolium iodide (30.5 g) as a waxy solid. Potassium 1,1,2,2-tetrafluoroethanesulfonate (TFES-K, 12 g) was added to reagent grade acetone (100 ml) in a separate round-bottomed flask, and this solution was carefully added to the 1-methyl-3-(3,3,4,4,5,5,6,6,7,7,8,8,8-tridecafluorooctyl)imidazolium iodide which had been dissolved in acetone (50 ml). The reaction mixture was heated under reflux for approximately 16 hours. The reaction mixture was then filtered using a large frit glass funnel to remove the white KI precipitate formed, and the filtrate was placed on a rotary evaporator for 4 hours to remove the acetone. The oily liquid was then filtered a second time to yield the product, as shown by proton NMR.

Examples 1-2 illustrate the formation of dibutyl ether from 1-butanol.

### Example 1

### Conversion of n-butanol to Dibutyl Ether

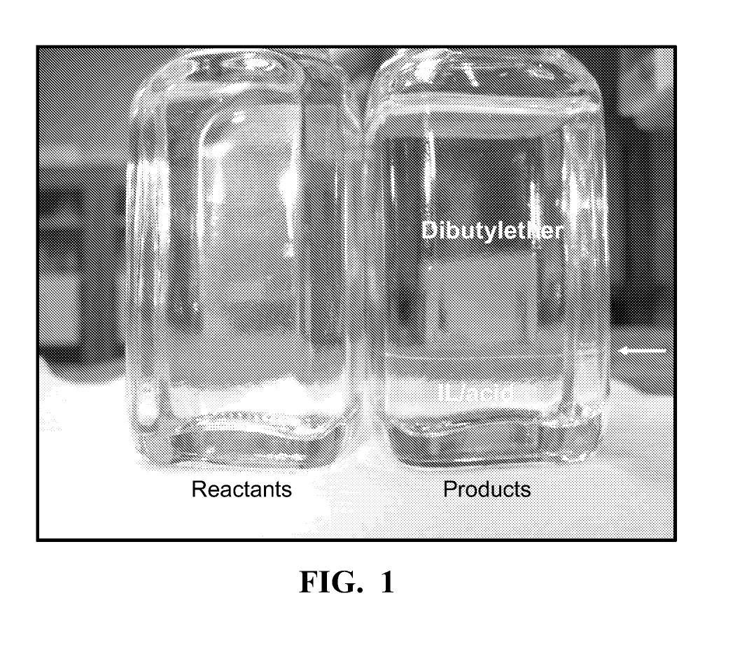

1-Butanol (30 g), 1-ethyl-3-methylimidazolium 1,1,2,2-tetrafluoroethanesulfonate (5 g), and 1,1,2,2-tetrafluoroethanesulfonic acid (0.6 g) were placed in a 200 ml shaker tube. The tube was heated under pressure with shaking for 6 h at 180° C. The vessel was then cooled to room temperature, and the pressure was released. Prior to heating the components were present as a single liquid phase (see FIG. 1 “Reactants”), however, the liquid became a 2-phase system (see FIG. 1 “Products”) after reacting and cooling the components. The top phase was shown by proton NMR to contain predominantly di-n-butyl ether with less than 10% 1-butanol. The bottom phase was shown to contain tetrafluoroethanesulfonic acid, 1-ethyl-3-methylimidazolium tetrafluoroethanesulfonate, and water. The conversion of 1-butanol was estimated to be about 90% by NMR. The two liquid phases were very distinct and separated within several minutes (<5 min).

### Example 2

### Conversion of n-butanol to Dibutyl Ether

1-Butanol (60 g), 1-ethyl-3-methylimidazolium 1,1,2,2-tetrafluoroethanesulfonate (10 g), and 1,1,2,2-tetrafluoroethanesulfonic acid (1.0 g) were placed in a 200 ml shaker tube. The tube was heated under pressure with shaking for 6 h at 180° C. Prior to heating the components were present as a single liquid phase. After reacting and cooling the components, the liquid became a 2-phase system, with a total weight of 58 g. The top phase was shown by proton NMR to contain greater than 75% dibutyl ether with less than 25% 1-butanol, and did not contain measurable quantities of ionic liquid or catalyst. The bottom phase was shown to contain 1,1,2,2-tetrafluoroethanesulfonic acid, 1-ethyl-3-methylimidazolium 1,1,2,2-tetrafluoroethanesulfonate, water and about 10% 1-butanol by weight relative to the combined weight of the ionic liquid, acid catalyst, water and butanol. The conversion of 1-butanol was estimated to be about 90%. The two liquid phases were very distinct and separated within several minutes (<5 min).

What is claimed is:

1. A process for preparing a dialkyl ether in a reaction mixture comprising (a) contacting at least one C₄ to C₈ straight-chain alcohol with at least one homogeneous acid catalyst in the presence of at least one ionic liquid to form (i) a dialkyl ether phase of the reaction mixture that comprises a dialkyl ether, and (ii) an ionic liquid phase of the reaction mixture; and (b) separating the dialkyl ether phase of the reaction mixture from the ionic liquid phase of the reaction mixture to recover a dialkyl ether product; wherein an ionic liquid is represented by the structure of the Formula Z⁺A⁻, wherein Z⁺ is a cation selected from the group consisting of: 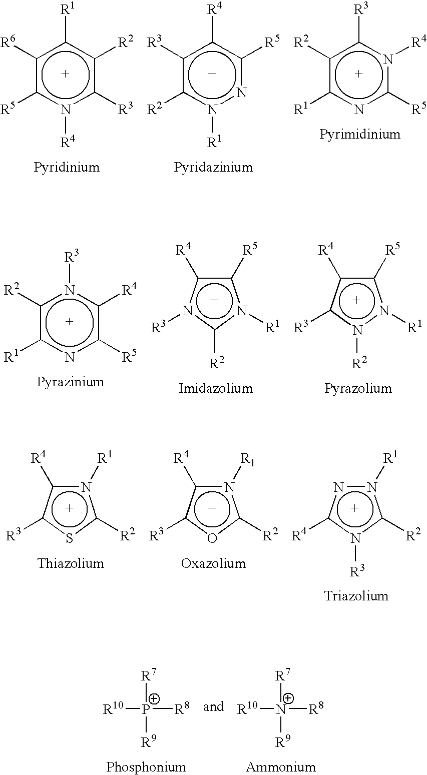
wherein R¹, R², R³, R⁴, R⁵ and R⁶ are independently selected from the group consisting of:
(i) H
(ii) halogen
(iii) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH;
(iv) —CH₃, —C₂H₅, or C₃ to C₂₅ straight-chain, branched or cyclic alkane or alkene comprising one to three heteroatoms selected from the group consisting of O, N, Si and S, and optionally substituted with at least one member selected from the group consisting of Cl, Br, F, I, OH, NH₂ and SH;
(v) C₆ to C₂₅ unsubstituted aryl or unsubstituted heteroaryl having one to three heteroatoms independently selected from the group consisting of O, N, Si and S; and
(vi) C₆ to C₂₅ substituted aryl or substituted heteroaryl having one to three heteroatoms independently selected from the group consisting of O, N, Si and S; and wherein said substituted aryl or substituted heteroaryl has one to three substituents independently selected from the group consisting of
(1) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH,
(2) OH,
(3) NH₂, and
(4) SH;
R⁷, R⁸, R⁹, and R¹⁰ are independently selected from the group consisting of:
(vii) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH;
(viii) —CH₃, —C₂H₅, or C₃ to C₂₅ straight-chain, branched or cyclic alkane or alkene comprising one to three heteroatoms selected from the group consisting of O, N, Si and S, and optionally substituted with at least one member selected from the group consisting of Cl, Br, F, I, OH, NH₂ and SH;
(ix) C₆ to C₂₅ unsubstituted aryl, or C₃ to C₂₅ unsubstituted heteroaryl having one to three heteroatoms independently selected from the group consisting of O, N, Si and S; and
(x) C₆ to C₂₅ substituted aryl, or C₃ to C₂₅ substituted heteroaryl having one to three heteroatoms independently selected from the group consisting of O, N, Si and S; and wherein said substituted aryl or substituted heteroaryl has one to three substituents independently selected from the group consisting of
(1) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH,
(2) OH,
(3) NH₂, and
(4) SH;
wherein optionally at least two of R¹, R², R³, R⁴, R⁵, R⁶, R⁷, R⁸, R⁹, and R¹⁰ can together form a cyclic or bicyclic alkanyl or alkenyl group; and
wherein A⁻ is an anion selected from the group consisting of R¹¹—SO₃⁻ and (R²²—SO₂)₂N⁻, and wherein R¹¹ and R¹² are independently selected from the group consisting of:
(a) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH;
(b) —CH₃, —C₂H₅, or C₃ to C₂₅ straight-chain, branched or cyclic alkane or alkene comprising one to three heteroatoms selected from the group consisting of O, N, Si and S, and optionally substituted with at least one member selected from the group consisting of Cl, Br, F, I, OH, NH₂ and SH;
(c) C₆ to C₂₅ unsubstituted aryl or unsubstituted heteroaryl having one to three heteroatoms independently selected from the group consisting of O, N, Si and S; and
(d) C₆ to C₂₅ substituted aryl or substituted heteroaryl having one to three heteroatoms independently selected from the group consisting of O, N, Si and S; and wherein said substituted aryl or substituted heteroaryl has one to three substituents independently selected from the group consisting of:
(1) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH,
(2) OH,
(3) NH₂, and
(4) SH.

2. The process of claim 1 wherein A⁻ is selected from the group consisting of [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻. [CF₃CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, and [(CF₂HCF₂SO₂)₂N]⁻, and [(CF₃CFHCF₂SO₂)₂N]⁻.

3. The process of claim 1 wherein an ionic liquid is selected from the group consisting of 1-butyl-2,3-dimethylimidazolium 1,1,2,2-tetrafluoroethanesulfonate, 1-butyl-methylimidazolium 1,1,2,2-tetrafluoroethanesulfonate, 1-ethyl-3-methylimidazolium 1,1,2,2-tetrafluoroethanesulfonate, 1-ethyl-3-methylimidazolium 1,1,2,3,3,3-hexafluoropropanesulfonate, 1-hexyl-3-methylimidazolium 1,1,2,2-tetrafluoroethanesulfonate, 1-dodecyl-3-methylimidazolium 1,1,2,2-tetrafluoroethanesulfonate, 1-hexadecyl-3-methylimidazolium 1,1,2,2-tetrafluoroethanesulfonate, 1-octadecyl-3-methylimidazolium 1,1,2,2-tetrafluoroethanesulfonate, N-(1,1,2,2-tetrafluoroethyl)propylimidazole 1,1,2,2-tetrafluoroethanesulfonate, N-(1,1,2,2-tetrafluoroethyl)ethylperfluorohexylimidazole 1,1,2,2-tetrafluoroethanesulfonate, 1-butyl-3-methylimidazolium 1,1,2,3,3,3-hexafluoropropanesulfonate, 1-butyl-3-methylimidazolium 1,1,2-trifluoro-2-(trifluoromethoxy)ethanesulfonate, 1-butyl-3-methylimidazolium 1,1,2-trifluoro-2-(perfluoroethoxy)ethanesulfonate, tetradecyl(tri-n-hexyl)phosphonium 1,1,2-trifluoro-2-(perfluoroethoxy)ethanesulfonate, tetradecyl(tri-n-butyl)phosphonium 1,1,2,3,3,3-hexafluoropropanesulfonate, tetradecyl(tri-n-hexyl)phosphonium 1,1,2-trifluoro-2-(trifluoromethoxy)ethanesulfonate, 1-ethyl-3-methylimidazolium 1,1,2,2-tetrafluoro-2-(pentafluoroethoxy)sulfonate, (3,3,4,4,5,5,6,6,7,7,8,8,8-tridecafluorooctyl)-trioctylphosphonium 1,1,2,2-tetrafluoroethanesulfonate, 1-methyl-3-(3,3,4,4,5,5,6,6,7,7,8,8,8-tridecafluorooctyl)imidazolium 1,1,2,2-tetrafluoroethanesulfonate, and tetra-n-butylphosphonium 1,1,2-trifluoro-2-(perfluoroethoxy)ethanesulfonate.

4. The process of claim 1 wherein a homogeneous acid catalyst has a pKa of less than about 4.

5. The process of claim 1 wherein the reaction mixture comprises an ionic liquid in an amount of about 0.1% or more, and yet in an amount of about 25% or less, by weight relative to the weight of the C₄ to C₈ alcohol present therein.

6. The process of claim 1 wherein a homogeneous acid catalyst is selected from the group consisting of inorganic acids, organic sulfonic acids, heteropolyacids, fluoroalkyl sulfonic acids, metal sulfonates, metal trifluoroacetates, compounds thereof and combinations thereof.

7. The process of claim 1 wherein a homogeneous acid catalyst is selected from the group consisting of sulfuric acid, fluorosulfonic acid, phosphorous acid, p-toluenesulfonic acid, benzenesulfonic acid, phosphotungstic acid, phosphomolybdic acid, trifluoromethanesulfonic acid, nonafluorobutanesulfonic acid, 1,1,2,2-tetrafluoroethanesulfonic acid, 1,1,2,3,3,3-hexafluoropropanesulfonic acid, bismuth triflate, yttrium triflate, ytterbium triflate, neodymium triflate, lanthanum triflate, scandium triflate, and zirconium triflate.

8. The process of claim 1 wherein the reaction mixture comprises a catalyst in an amount of about 0.1% or more, and yet in an amount of about 20% or less, by weight relative to the weight of the C₄ to C₈ alcohol present therein.

9. The process of claim 1 wherein the C₄ to C₈ straight-chain alcohol is selected from the group consisting of n-butanol, n-pentanol, n-hexanol, n-heptanol and n-octanol.

10. The process of claim 1 wherein the C₄ to C₈ straight-chain alcohol is n-butanol and said dialkyl ether is dibutyl ether.

11. The process of claim 1 which is carried out under an inert atmosphere.

12. The process of claim 1 wherein the dialkyl ether product is in the vapor phase.

13. The process of claim 1 wherein the ionic liquid phase comprises catalyst residue.

14. The process of claim 1 wherein the separated ionic liquid phase is recycled to the reaction mixture.

15. The process of claim 1 wherein water is removed from the separated ionic liquid phase.

16. The process of claim 1 wherein the C₄ to C₈ straight-chain alcohol is n-butanol, wherein the reaction occurs at a temperature of from about 50 degrees C. to about 300 degrees C. and at a pressure of from about 0.1 MPa to about 20.7 MPa.

17. The process of claim 1 wherein the C₄ to C₈ straight-chain alcohol is n-butanol, wherein the reaction occurs at a temperature of from about 50 degrees C. to about 300 degrees C. and at a pressure of from about 0.1 MPa to about 20.7 MPa, and wherein an ionic liquid is 1-ethyl-3-methylimidazolium 1,1,2,2-tetrafluoroethanesulfonate.

18. The process of claim 1 wherein the C₄ to C₈ straight-chain alcohol is n-butanol, wherein the reaction occurs at a temperature of from about 50 degrees C. to about 300 degrees C. and at a pressure of from about 0.1 MPa to about 20.7 MPa, wherein an ionic liquid is 1-ethyl-3-methylimidazolium 1,1,2,2-tetrafluoroethanesulfonate, and wherein a homogeneous acid catalyst is 1,1,2,2-tetrafluoroethanesulfonic acid.
